(19) United States
(12) Patent Application Publication
(10) Pub. No.: US 2021/0011046 A1
(43) Pub. Date: Jan. 14, 2021

(54) SAMPLE PROCESSING AND DETECTING KIT WITH A MATERIAL TRANSFER STRUCTURE

(71) Applicant: Merlin Biomedical (Xiamen) Co., Ltd., Xiamen (CN)

(72) Inventors: Yifeng SHEN, Xiamen (CN); Lijin ZHENG, Xiamen (CN)

(21) Appl. No.: 17/015,694

(22) Filed: Sep. 9, 2020

Related U.S. Application Data
(63) Continuation of application No. PCT/CN2019/081482, filed on Apr. 4, 2019.

(30) Foreign Application Priority Data
Apr. 4, 2018 (CN) 201810299146.9

(51) Int. Cl. G01N 35/10; B01L 3/00; G01N 35/02

(52) U.S. Cl. CPC G01N 35/1079; B01L 3/502; G01N 35/025; B01L 2200/16; G01N 2035/1088; B01L 2300/0803; B01L 2300/0832; B01L 2400/0487; B01L 2300/044

(57) ABSTRACT

A sample processing and detecting kit with a material transfer structure comprises a kit body defined by an outer shell. A top outer shell of the kit body comprises an opening-and-closing sample port. An inner side of the kit body is disposed with a material circular plate holder and a middle of the material circular plate holder is disposed with a circular hole. A waste liquid storage chamber, a first lock groove, a second lock groove, and a sealing hole are arranged on the material circular plate holder at a periphery of the circular hole. A sample processing plastic tube member is hermitically disposed in the first lock groove. A detection reaction plastic tube member is hermetically disposed in the second lock groove.

### RELATED APPLICATIONS

This application is a continuation of and claims priority to International Patent Application PCT/CN2019/081482, filed on Apr. 4, 2019, which claims priority to Chinese Patent Application 201810299146.9, filed on Apr. 4, 2018. International Patent Application PCT/CN2019/081482 and Chinese Patent Application 201810299146.9 are incorporated herein by reference.

### FIELD OF THE DISCLOSURE

The present disclosure relates to the technical field of sample processing and detecting consumables, and in particular relates to a sample processing and detecting kit with a material transfer structure.

### BACKGROUND OF THE DISCLOSURE

At present, the conventional molecular detection technology platforms mainly comprise nucleic acid amplification technology, molecular hybridization technology, Deoxyribonucleic acid (DNA) sequencing technology, and biochip technology. The main applications of molecular detection in medical tests are infectious detection of pathogenic microorganisms, diagnosis of tumors and genetic diseases, diagnosis of immune system diseases, prenatal screening, etc., as well as in medical examination centers, technical service centers, and third-party detection agencies. With changes in the medical model and the continuous development of personalized medicine, the medical detection community urgently needs fast and accurate detection methods, and molecular detection has played a unique advantage.

The mainstream technology of molecular detection is fluorescent quantitative polymerase chain reaction (PCR) technology. Due to the characteristics of the exponential amplification template of PCR technology, aerosol contamination caused by open consumables has become an important factor limiting the further application of fluorescent quantitative PCR technology to clinical applications. Traditional products have unstable results and are difficult to implement multiple detection due to complicated experimental steps and operations, PCR aerosol contamination, low degree of automation, and manual operation.

Molecular detection technology is developing in the direction of accuracy, convenience, sensitivity, automation, and integration, but due to the technical complexity of molecular detection itself, there are very few fully automated, integrated instrument platforms from samples to results. There are many difficult problems such as complex structure, poor sensitivity, complicated operation, and complicated detection equipment.

### BRIEF SUMMARY OF THE DISCLOSURE

The present disclosure provides a sample processing and detecting kit with a material transfer structure to solve deficiencies of the existing techniques.

In order to solve the aforementioned technical problems, the present disclosure provides a first technical solution.

A sample processing and detecting kit with a material transfer structure comprises a kit body defined by an outer shell. A top outer shell of the kit body comprises an opening-and-closing sample port. An inner side of the kit body is disposed with a material circular plate holder and a middle of the material circular plate holder is disposed with a circular hole. A waste liquid storage chamber, a first lock groove, a second lock groove, and a sealing hole are arranged on the material circular plate holder at a periphery of the circular hole. A sample processing plastic tube member is hermitically disposed in the first lock groove. A detection reaction plastic tube member is hermetically disposed in the second lock groove. The material circular plate holder is rotatably connected to the material transfer structure through the circular hole. The material transfer structure comprises a hollow rotor disposed with a pipette tip, and a bottom end of the hollow rotor is disposed with a hollow pointed structure for piercing a sealing film. A rotatable connection structure surrounds a bottom end of the hollow rotor. A silicone seal disposed below the bottom end of the hollow rotor is disposed in the rotatable connection structure. A spring surrounds an outer side of the hollow rotor, a lower side of the spring is disposed with a first seal ring surrounding an outer side of a bottom end of the material transfer structure, and an upper portion of the material transfer structure is hermetically disposed in the kit body through the first seal ring.

In a preferred embodiment, a second fixing buckle is disposed on a side wall of the circular hole, an upper end portion of the hollow rotor is disposed with a first fixing buckle configured to be buckled to the second fixing buckle, and a pressure ring is disposed below the first seal ring and above the rotatable connection structure.

In a preferred embodiment, the kit body is a hollow cylindrical structure with a hollowed bottom, a hollow square cylindrical structure with a hollowed bottom, a hollow polygonal cylindrical structure with a hollowed bottom, or a hollow irregular cylindrical structure with a hollowed bottom, and the material circular plate holder is a plate body adapted to the kit body.

In a preferred embodiment, the material circular plate holder is a plastic structure integrally formed. An outer circumference of the material circular plate holder is disposed with a second seal ring, and the material circular plate holder is hermetically connected to the kit body to define a closed extraction and detection reaction space by the second seal ring.

In a preferred embodiment, the sample processing plastic tube member is disposed with multiple reagent storage chambers and a reaction chamber, and the reagent storage chambers and the reaction chamber define a closed area through the sealing film.

In a preferred embodiment, the detection reaction plastic tube member comprises a plurality of detection reaction chambers, and inner sides of the plurality of detection reaction chambers store detection reaction reagents or detection reaction lyophilized powders configured to complete a detection reaction of one or more items.

In a preferred embodiment, the material transfer structure comprises a hollow rotatable shaft. The pipette tip extends and overhangs from an end of the hollow rotatable shaft, and the pipette tip comprises the hollow pointed structure for piercing the sealing film.

In a preferred embodiment, the hollow rotatable shaft comprises a first through hole, a first end of the first through hole is in communication with the pipette tip, and a second end of the first through hole penetrates the hollow rotatable shaft and abuts the silicone seal disposed in the rotatable connection structure. An anti-backflow mechanism is disposed on a joint of the hollow rotatable shaft and the pipette tip.

In a preferred embodiment, the rotatable connection structure is engaged with an external device to enable the hollow rotor to rotate and to enable liquid to be sucked in and to be blown out.

The present disclosure provides a second technical solution.

A sample processing and detecting kit with a material transfer structure comprises a kit body defined by an outer shell, a material plate holder, and the material transfer structure. A top outer shell of the kit body comprises an opening-and-closing sample port. A middle of the material plate holder is disposed with a circular hole. A periphery of the circular hole is disposed with a waste liquid storage chamber, a first lock groove, a second lock groove, and a sealing hole. A sample processing plastic tube member is hermetically disposed in the first lock groove, and a detection reaction plastic tube member is hermetically disposed in the second lock groove. The material transfer structure comprises a hollow rotor disposed with a pipette tip, a rotatable connection structure, and a first seal ring. An upper end portion of the hollow rotor is disposed with a hollow pointed structure for piercing a sealing film. The material plate holder is disposed in the kit body. The material plate holder is rotatably connected to the material transfer structure through the circular hole. A rotatable connection structure surrounds a bottom end or an upper portion of the hollow rotor, and a silicone seal disposed below the bottom end of the hollow rotor is disposed in the rotatable connection structure. The first seal ring surrounds an outer side of the hollow rotor, the first seal ring is disposed in the circular hole, and an upper portion of the material transfer structure is hermetically disposed in the kit body through the first seal ring. The rotatable connection structure drives the hollow rotor to rotate relative to the material plate holder, and the hollow rotor is configured to move upward and downward relative to the material plate holder.

In a preferred embodiment, the hollow rotor comprises a hollow rotatable shaft, and the pipette tip extends and suspends from the upper portion of the hollow rotatable shaft.

In a preferred embodiment, the hollow rotatable shaft is disposed with a first through hole, a first end of the first through hole is in communication with the pipette tip, and a second end of the first through hole penetrates the hollow rotatable shaft and abuts the silicone seal disposed in the rotatable connection structure. An inner side of the hollow rotatable shaft is disposed with a filter at a joint of the hollow rotatable shaft and the pipette tip.

In a preferred embodiment, a reinforcing rib is disposed in the first lock groove, and the sample processing plastic tube member is clamped in the first lock groove.

In a preferred embodiment, a reinforcing rib is disposed in the second lock groove to define a plurality of lock groove units, the detection reaction plastic tube member comprises a plurality of detection reaction chambers, and each of the plurality of detection reaction chambers are clamped in a corresponding one of the plurality of lock groove units.

In a preferred embodiment, the sealing hole is an individual tube clamped in one of the plurality of lock groove units or a sample processing tube of the sample processing plastic tube member.

In a preferred embodiment, the rotatable connection structure is disposed on the upper end portion of the hollow rotor, and the pipette tip is disposed below the rotatable connection structure. The kit body comprises an assembly hole, and the upper end portion of the hollow rotor penetrates out of the assembly hole of the kit body to surround the rotatable connection structure.

In the present disclosure, the sample processing and detecting reaction with material transferring is formed in the kit body, so nucleic acids or proteins can be prepared and detected in a closed space. Compared with the existing open environment and operation performed manually step-by-step, the operation is not only simple and convenient, but also can integrate the preparation and the detection of biological macromolecules such as nucleic acids or proteins to overcome the problems of tedious operation, being time-consuming and labor-intensive, and sample or reagent contamination in the steps of extraction, detection, and interconnection between the extraction and the detection, In addition, all extraction and detection steps can be performed in the same closed space, so that a probability of sample contamination is reduced, and an accuracy of sample extraction and detection is improved. Further, the present disclosure comprises a unique material transfer structure that allows materials to be transferred at any arc comprising an angle within 360 degree in the sample processing plastic tube member, the detection reaction plastic tube member, and the waste liquid storage chamber, which greatly simplifies the overall structure.

### BRIEF DESCRIPTION OF THE DRAWING

FIG. 1 illustrates a cross-sectional view of Embodiment 1 according to the present disclosure;

FIG. 2 illustrates a schematic view of a material circular plate holder of Embodiment 1 according to the present disclosure;

FIG. 3 illustrates a schematic view of a material transfer structure of Embodiment 1 according to the present disclosure;

FIG. 4 illustrates a schematic view of a sample processing plastic tube member of Embodiment 1 according to the present disclosure;

FIG. 5 illustrates a schematic view of a sample reaction plastic pipe of Embodiment 1 according to the present disclosure;

FIG. 6 illustrates an exploded perspective view of Embodiment 2 according to the present disclosure;

FIG. 7 illustrates a cross-sectional view of Embodiment 2 according to the present disclosure in an initial state;

FIG. 8 illustrates a schematic view of an internal structure of Embodiment 2 according to the present disclosure in the initial state;

FIG. 9 illustrates a cross-sectional view of Embodiment 2 according to the present disclosure in a termination state;

FIG. 10 illustrates a schematic view of the internal structure of Embodiment 2 according to the present disclosure in the termination state;

FIG. 11 illustrates a schematic view of a material plate holder of Embodiment 2 according to the present disclosure;

FIG. 12 illustrates a schematic view of a single tube of a detection reaction plastic tube member of Embodiment 2 according to the present disclosure;

FIG. 13 illustrates a schematic view of a sample processing plastic tube member of Embodiment 2 according to the present disclosure; and

FIG. 14 illustrates a schematic view of a material transfer structure of Embodiment 2 according to the present disclosure.

### DETAILED DESCRIPTION OF THE EMBODIMENTS

The present disclosure will be further described with the combination of the accompanying embodiments and the accompanying drawings. It is apparent that the embodiments described below are merely a part of the embodiments of the present disclosure, but not all of the embodiments.

### Embodiment 1

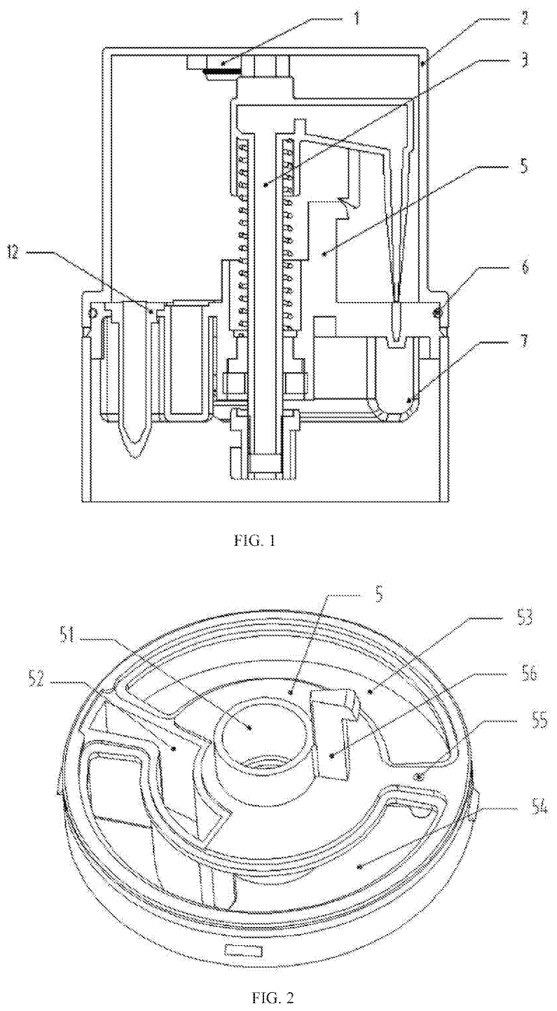

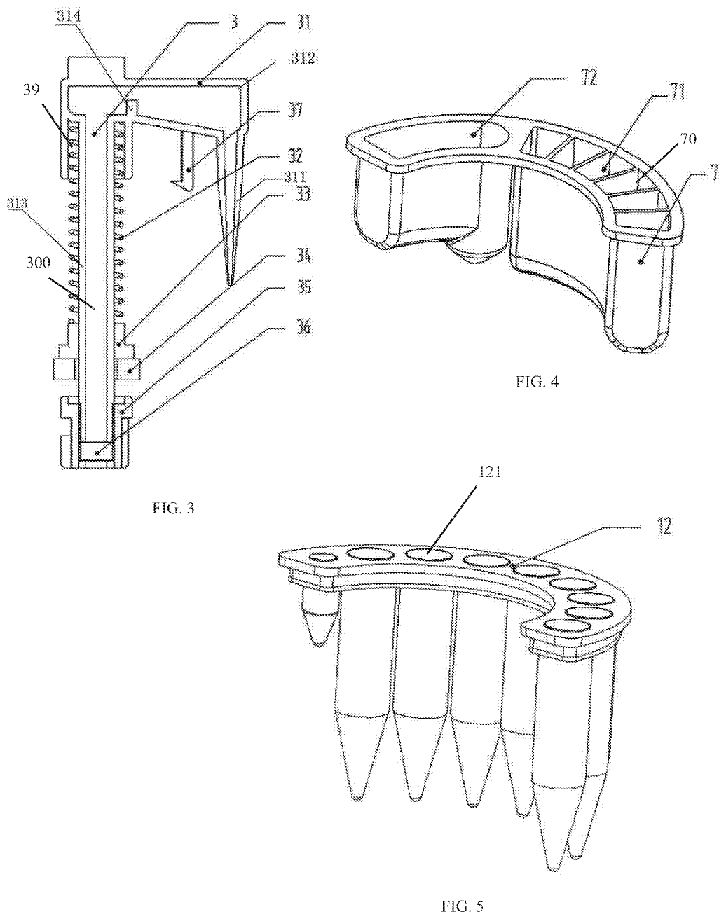

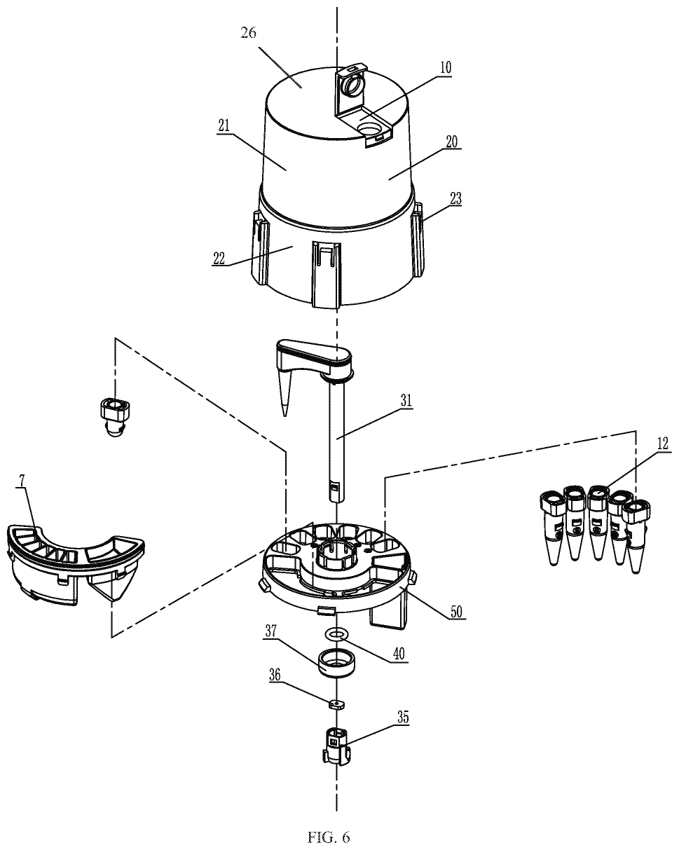

Referring to FIGS. 1-5, a sample processing and detecting kit with a material transfer structure comprises a kit body 2 defined by an outer shell, and a top outer shell of the kit body 2 comprises an opening-and-closing sample port 1. A material circular plate holder 5 is disposed in the kit body 2, and a circular hole 51 is disposed on a middle of the material circular plate holder 5. A second fixing buckle 56 is disposed on a side wall of the circular hole 51. A waste liquid storage chamber 52, a first lock groove 53, a second lock groove 54, and a sealing hole 55 are disposed on the material circular plate holder 5 at a periphery of the circular hole 51. A sample processing plastic tube member 7 is hermetically disposed in the first lock groove 53, and a detection reaction plastic tube member 12 is hermetically disposed in the second lock groove 54.

The material transfer structure 3 comprises a hollow rotor 31 disposed with a pipette tip 312, and an end portion of the hollow rotor 31 is disposed with a hollow pointed structure 311 for piercing a sealing film and a first fixing buckle 37 configured to be locked to the second fixing buckle 56. A rotatable connection structure 35 surrounds a bottom end of the hollow rotor 31, and a silicon seal 36 disposed below the bottom end of the hollow rotor 31 is disposed in the rotatable connection structure 35. A spring 32 surrounds an outer side of the hollow rotor 31, and a lower side of the spring 32 is disposed with a first seal ring 33 and a pressure ring 34 that surround an outer side a bottom end of the material transfer structure 3. The first seal ring 33 is disposed above the pressure ring 34, and the pressure ring 34 is disposed above the rotatable connection structure 35. The upper portion of the material transfer structure 3 is hermetically disposed in the kit body 2 by the first seal ring 33 and the pressure ring 34.

The kit body 2 is a hollow cylindrical structure with a hollowed bottom, a square column structure with a hollowed bottom, a polygonal column structure with a hollowed bottom, or an irregular column structure with a hollowed bottom. The material circular plate holder 5 is hermetically disposed in the kit body 2, and a shape of the material circular plate holder 5 corresponds to a shape of an internal structure of the kit body 2.

The material circular plate holder 5 is a plastic structure integrally formed, and an outer circumference of the material circular plate holder 5 is disposed with a second seal ring 6. The material circular plate holder 5 is hermetically connected to the kit body 2 to define a closed extraction and detection reaction space by the second seal ring 6. The sample processing plastic tube member 7 is disposed with multiple reagent storage chambers 71 and a reaction chamber 72. The multiple reagent storage chambers 71 and the reaction chamber 72 define a closed area through a sealing film 70. One of the multiple reagent storage chambers 71 and the reaction chamber 72 is configured to store magnetic particles or extraction reagent comprising magnetic particle, and a bottom of the reaction chamber 72 is connected to an external detection equipment.

The detection reaction plastic tube member 12 is disposed with a plurality of detection reaction chambers 121. Inner sides of the plurality of detection reaction chambers 121 store detection reaction reagents or detection reaction lyophilized powder configured to complete a detection reaction of one or more items, and the detection reaction plastic tube member 12 is a set of PCR tubes. The shape of the material transfer structure 3 is “7” shaped. An upper portion of the material transfer structure 3 is disposed with a lock groove 39, and one end of the spring 32 is clamped in the lock groove. The hollow rotor 31 comprises a hollow rotatable shaft 313, and an upper portion of the pipette tip 312 extends and suspends from the hollow rotatable shaft 313. The hollow rotatable shaft 313 is disposed with a first through hole 300. A first end of the first through hole 300 is in communication with the pipette tip 312, and a second end of the first through hole 300 penetrates the hollow rotatable shaft 313 and abuts a silicone seal 36 disposed in the rotatable connection structure 35. An anti-backflow mechanism 314 is disposed on a joint of the hollow rotatable shaft 313 and the pipette tip 312 to prevent liquid in the pipette tip 312 from flowing to the hollow rotatable shaft 313.

External devices comprise a stepping motor that drives the rotatable connection structure 35 to rotate and move upward and downward and a plunger pump for sucking in and blowing out liquid. The rotatable connection structure 35 is engaged with the external devices to enable the hollow rotor to rotate and to enable liquid to be sucked in and to be blown out through the external devices.

The working principle of the first embodiment is as follows. In the present disclosure, the external devices drive the hollow rotor 31 and the hollow pointed structure 311 to rotate to a required position, and then a downward force is applied to the hollow rotor 31 and the hollow pointed structure 311 by the external devices. When the hollow rotor 31 moves downward, the hollow pointed structure 311 will pierce a corresponding position of the sealing film. After the external devices are engaged with the rotatable connection structure 35, the pipette tip 312 sucks liquid reagent into or blows the liquid reagent out from the reaction chamber 72, the multiple reagent storage chambers 71, the plurality of detection reaction chambers 121, and the waste liquid storage chamber 52. When the downward force applied by the external devices is removed, the hollow rotor 31 moves back to its initial position under a spring force. At the same time, the material transfer structure 3 can allow materials to be transferred at any arc comprising an angle within 360 degree in the sample processing plastic tube member 7, the plurality of detection reaction chambers 121, and the waste liquid storage chamber 52, and overcomes the problems of tedious operation, being time-consuming and labor-intensive, and sample or reagent contamination in the steps of extraction, detection, and interconnection between the extraction and the detection. The present disclosure is used with convenience.

When performing nucleic acid extraction and PCR detection, magnetic particles comprising magnetic balls are used to extract biological macromolecular extracts such as nucleic acids and proteins. After the extraction in the reaction chamber 72, the magnetic particles in the reaction chamber 72 suck nucleic acids, proteins, and other biological macromolecular extracts. The magnetic particles are sucked on an inner wall of a side of the reaction chamber 72 by magnetic force of the external devices applied on the side of the reaction chamber 72. At this time, the liquid reagent can be effectively transferred to the waste liquid storage chamber 52 through the material transfer structure 3, and a cleaning solution in the multiple reagent storage chambers 71 is then sucked and transferred into the reaction chamber 72. At this time, the magnetic force of the external devices applied on the side of the reaction chamber 72 is removed, and then the external devices drive the material transfer structure 3 to suck in and blow out the cleaning solution to wash the magnetic particles sucked with the biological macromolecular extracts such as nucleic acids and proteins. Then the cleaning solution is transferred to the waste liquid storage chamber 52. Similarly, subsequent steps are also completed by the same process, by way of example, cleaning solution and eluent in the multiple reagent storage chambers 71 are transferred to the reaction chamber 72 to obtain the eluted extract solution, the eluted extract solution is transferred to the detection reaction plastic tube member 12. More specifically, one of the multiple reagent storage chambers 71 stores sterile water. Before biological macromolecular extracts such as nucleic acids or proteins is eluted, the pipette tip 312 of the material transfer structure 3 is washed in sterile water, then the elution step is performed to ensure that the eluted extract solution for biological macromolecular extract such as nucleic acids or proteins without impurities is obtained. After that, the elution solution is transferred to the detection reaction plastic tube member 12 and mixed with the detection reaction reagents or the detection reaction lyophilized powders in the detection reaction plastic tube member 12 by blowing in and sucking out. Then paraffin oil is transferred from the multiple reagent storage chambers 71 to seal the liquid. When the PCR detection reaction is performed, the pipette tip 312 of the hollow rotor 31 of the material transfer structure 3 moves above the sealing hole 55 of the material circular plate holder 5. The first fixing buckle 37 of the material transfer structure 3 is locked to the second fixing buckle 56 of the material circular plate holder 5 through a downward force, and the pipette tip 312 of the hollow rotor 31 enters into the sealing hole 55 of the material circular plate holder 5 to define a closed structure, so that the PCR reaction aerosol will not diffuse to outer side.

When performing an enzyme-linked immunoassay reaction, the multiple reagent storage chambers 71 store enzyme-linked immunoassay reagents, and the sample processing plastic tube member 7 stores microplates embedded with antibodies or antigens. The number and the shape of the multiple reagent storage chambers 71 and the number and the shape of the microplates embedded with antibodies or antigens of a detection reaction area (e.g., the detection reaction plastic tube member 12) are adjusted according to a detection purpose and item requirements. First, a sample is added through the opening-and-closing sample port 1 of the kit body 2, and the external devices are operatively connected to the material transfer structure 3 to drive the material transfer structure 3 to rotate, the sample is added into each of the microplates embedded with antibodies or antigens of the detection reaction plastic tube member 12. Then the detection reaction plastic tube member 12 is connected to an external heating device, and the microplates embedded with antibodies or antigens is incubated at a temperature set by the external heating device. Then, a washing solution in the multiple reagent storage chambers 71 is transferred into each of the microplates embedded with antibodies or antigens of the detection reaction area by the material transfer structure 3 to process washing. Subsequently, enzyme conjugate from the reagent storage chamber 71 are transferred into each of the microplates embedded with antibodies or antigens of the detection reaction area, the incubation at the temperature of each of the microplates embedded with antibodies or antigens of the detection reaction area, a washing solution in the multiple reagent storage chambers 71 is transferred into each of the microplates embedded with antibodies or antigens of the detection reaction area, the aforementioned processes are also completed by the same procedure. Further subsequently, a color developing solution in the reagent storage chamber 71 is transferred to each of the microplates embedded with antibodies or antigens by the material transfer structure 3, and is mixed for color development by the blowing in and sucking out of the material transfer structure 3. Finally, a final solution in the multiple reagent storage chambers 71 is transferred into each of the microplates embedded with antibodies or antigens by the material transfer structure 3 to be mixed and terminate the reaction. The microplates embedded with antibodies or antigens of the kit are connected to an external microplate reader to determine the optical density (OD) value of each of the microplates embedded with antibody or antigens.

### Embodiment 2

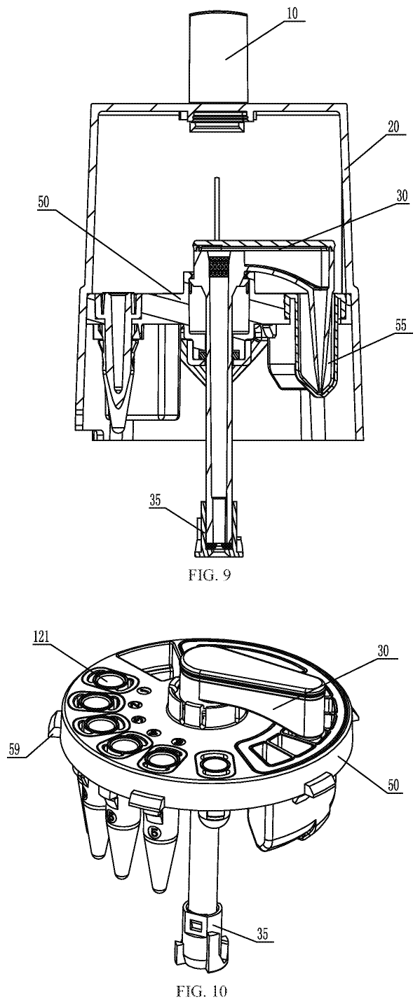

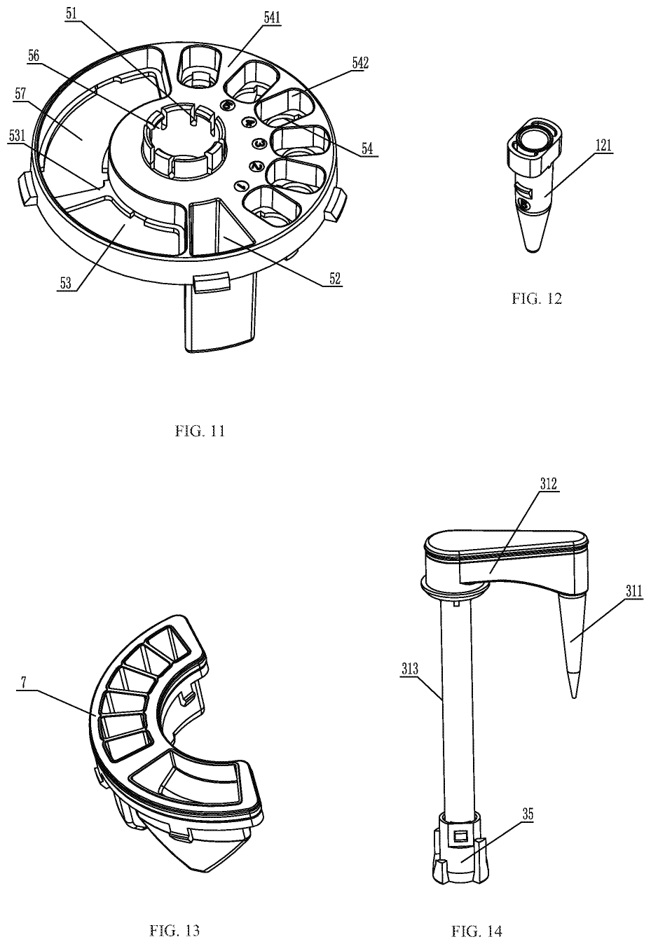

Referring to FIGS. 6-12, a sample processing and detecting kit with a material transfer structure comprises a kit body 20 defined by an outer shell, a material plate holder 50, a material transfer structure 30, a first seal ring 40, and a second seal ring 6.

A top outer shell 26 of the kit body 20 comprises an opening-and-closing sample port 1.

A middle of the material plate holder 50 comprises a circular hole 51, and a waste liquid storage chamber 52, a first lock groove 53, a second lock groove 54, and a sealing hole 55 are disposed on a periphery of the circular hole 51. A sample processing plastic tube member 7 is hermetically disposed in the first lock groove 53, and the detection reaction plastic tube member 12 is hermetically disposed in the second lock groove 54. A reinforcing rib 531 is disposed in the first lock groove 53 to define a third lock groove 57.

The material transfer structure 30 comprises a hollow rotor 31 disposed with a pipette tip 312, a rotatable connection structure 35, the first seal ring 40, and a fixing sleeve 38.

The rotatable connection structure 35 surrounds a bottom end of the hollow rotor 31, and a silicone seal 36 disposed below the bottom end of the hollow rotor 31 is disposed in the rotatable connection structure 35. The first seal ring 40 surrounds an outer side of the hollow rotor 31. The first seal ring 40 is disposed in the circular hole 51, and an upper portion of the material transfer structure 30 is hermetically disposed in the kit body 20 through the first seal ring 40.

The rotatable connection structure 35 drives the hollow rotor 31 to rotate relative to the material plate holder 50, and the hollow rotor 31 is configured to move upward and downward relative to the material plate holder 50.

In this embodiment, the kit body 20 is a hollow cylindrical structure with a hollowed bottom. The kit body 20 comprises an upper cylinder portion 21 and a lower cylinder portion 22. A diameter of the upper cylinder portion 21 is smaller than a diameter of the lower cylinder portion 22 to define a positioning protrusion platform 24. A cylinder wall of the lower cylinder portion 22 comprises a plurality of elastic lock grooves 23.

In this embodiment, the hollow rotor 31 comprises a hollow rotatable shaft 313. The pipette tip 312 extends and suspends from an upper portion of the hollow rotatable shaft 313. A lower end portion of the pipette tip 312 comprises a hollow pointed structure 311 for piercing a sealing film. The hollow rotatable shaft 313 is comprises a first through hole 300. A first end of the first through hole 300 is in communication with the pipette tip 312, and a second end of the first through hole 300 penetrates the hollow rotatable shaft 313 and abuts the silicone seal 36 disposed in the rotatable connection structure 35. A filter 315 is disposed on a joint of the hollow rotatable shaft 313 and the pipette tip 312 to prevent liquid in the pipette tip 312 from flowing to the hollow rotatable shaft 313.

In this embodiment, the material plate holder 50 is a plastic structure integrally formed, and an elastic clamping block 59 is disposed on an outer peripheral surface of a lower portion of the material plate holder 50. The circular hole 51 disposed in the middle comprises an upper positioning protrusion platform 511 protruding from an upper end surface of the material plate holder 50 and a lower positioning protrusion platform 512 protruding from a lower end surface of the material plate holder 50. An inner hole of the circular hole 51 is a stepped hole. The first seal ring 40 is disposed in a large portion of the stepped hole comprising a first diameter, and an outer diameter of the hollow rotatable shaft 313 cooperates with a small portion of the stepped hole with a second diameter, and the first diameter is larger than the second diameter. A lower portion of the hollow rotatable shaft 313 penetrates out from the small portion through the first seal ring 40, and the fixing sleeve 38 surrounds the lower positioning protrusion platform of the material plate holder 50. The first seal ring 40 is an O-ring.

The material plate holder 50 is disposed in the kit body 20, and the upper end surface of the material plate holder 50 abuts the positioning protrusion platform 24 in the kit body 20. The elastic clamping block 59 disposed on the outer wall of the material plate holder 50 is fixedly clamped to the plurality of elastic lock grooves 23 disposed in the kit body 20. The upper end surface of the material plate holder 50 is sealed with the kit body 20 to define a closed extraction and detection reaction space, and the pipette tip 312 of the upper portion of the hollow rotatable shaft 313 is disposed in the closed extraction and detection reaction space.

In the present embodiment, a reinforcing rib 541 is disposed in the second lock groove 54 to define a plurality of lock groove units 542. Correspondingly, the detection reaction plastic tube member 12 comprises a plurality of individual tubes (e.g., detection reaction chambers 121), each of the plurality of individual tubes are clamped in a corresponding one of the plurality of lock groove units 542.

The external devices comprises a stepping motor that drives the rotatable connection structure 35 to rotate and move upward and downward and a plunger pump for sucking in and blowing out liquid. The rotatable connection structure 35 is engaged with the external devices to enable the hollow rotor to rotate and to enable liquid to be sucked in and to be blown out through the external devices.

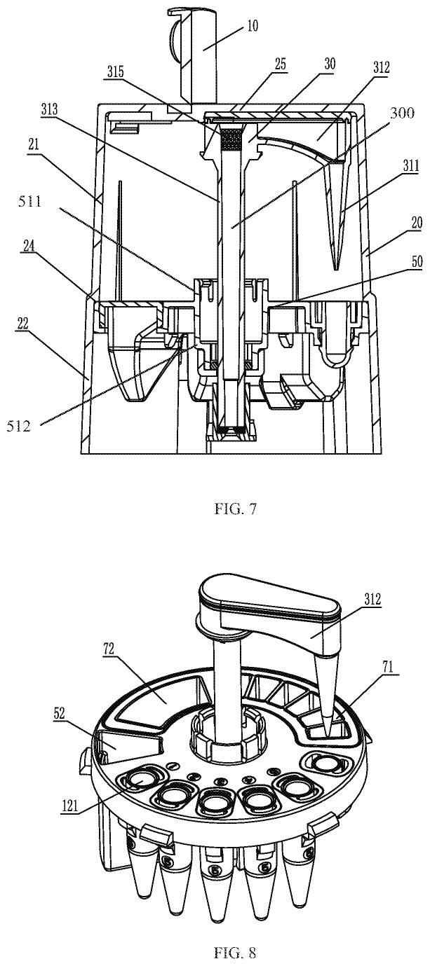

Referring to FIGS. 7-10, when the present disclosure is at an initial position, the top of the material transfer structure 30 is locked to a positioning slot 25 of the kit body 20. A hollow pointed structure 311 of a lower end of the pipette tip 312 is spaced away from the upper end surface of the material plate holder 50 at a safe distance. When in use, the stepping motor drives the hollow rotor 31 to rotate or move upward and downward through the rotatable connection structure 35, and the pipette tip 312 works. At the same time, the plunger pump is in communication with the silicone seal 36 disposed below the bottom end of the hollow rotor 31 to suck in and blow out liquid. At the end of the operation, the stepping motor drives the rotatable connection structure 35 to drive the hollow rotor 31 to rotate until the pipette tip 312 is located above the sealing hole 55, and then the hollow rotor 31 moves downward until the hollow pointed structure 311 is inserted into and locked to the sealing hole 55.

In the present disclosure, the sealing hole can be a single tube clamped in one of the plurality of lock groove units 542. The sealing hole can also be a tube of the sample processing plastic tube member 7.

In the present disclosure, the rotatable connection structure 35 can be disposed on the upper end portion of the hollow rotor 31. Correspondingly, the pipette tip 312 is disposed below the rotatable connection structure, the kit body 20 comprises an assembly hole, and the upper end portion of the hollow rotor 31 penetrates out of the assembly hole disposed on the kit body 20 to surround the rotatable connection structure 35.

It will be apparent to those skilled in the art that various modifications and variation can be made in the present disclosure without departing from the spirit or scope of the disclosure. Thus, it is intended that the present disclosure cover the modifications and variations of this disclosure provided they come within the scope of the appended claims and their equivalents.

### INDUSTRIAL APPLICABILITY

The present disclosure provides a sample processing and detecting kit with a material transfer structure. The present disclosure comprises a unique material transfer structure that allows materials to be transferred at any arc comprising an angle within 360 degree in the sample processing plastic tube member, the detection reaction plastic tube member, and the waste liquid storage chamber, which greatly simplifies the overall structure, a probability of sample contamination is reduced, and an accuracy of sample extraction and detection is improved. An application range is wide, and an industrial applicability is good.

What is claimed is:

1. A sample processing and detecting kit with a material transfer structure, comprising:
a kit body defined by an outer shell, wherein:
a top outer shell of the kit body comprises an opening-and-closing sample port,
an inner side of the kit body is disposed with a material circular plate holder,
a middle of the material circular plate holder is disposed with a circular hole,
a waste liquid storage chamber, a first lock groove, a second lock groove, and a sealing hole are arranged on the material circular plate holder at a periphery of the circular hole,
a sample processing plastic tube member is hermitically disposed in the first lock groove,
a detection reaction plastic tube member is hermetically disposed in the second lock groove,
the material circular plate holder is rotatably connected to the material transfer structure through the circular hole,
the material transfer structure comprises a hollow rotor disposed with a pipette tip,
a bottom end of the hollow rotor is disposed with a hollow pointed structure for piercing a sealing film,
a rotatable connection structure surrounds a bottom end of the hollow rotor,
a silicone seal disposed below the bottom end of the hollow rotor is disposed in the rotatable connection structure,
a spring surrounds an outer side of the hollow rotor,
a lower side of the spring is disposed with a first seal ring surrounding an outer side of a bottom end of the material transfer structure, and
an upper portion of the material transfer structure is hermetically disposed in the kit body through the first seal ring.

2. The sample processing and detecting kit with the material transfer structure according to claim 1, wherein:
a second fixing buckle is disposed on a side wall of the circular hole,
an upper end portion of the hollow rotor is disposed with a first fixing buckle configured to be buckled to the second fixing buckle, and
a pressure ring is disposed below the first seal ring and above the rotatable connection structure.

3. The sample processing and detecting kit with the material transfer structure according to claim 1, wherein:
the kit body is a hollow cylindrical structure with a hollowed bottom, a hollow square column structure with a hollowed bottom, a hollow polygonal column structure with a hollowed bottom, or a hollow irregular column structure with a hollowed bottom, and
the material circular plate holder is a plate body adapted to the kit body.

4. The sample processing and detecting kit with the material transfer structure according to claim 1, wherein:
the material circular plate holder is a plastic structure integrally formed,
an outer circumference of the material circular plate holder is disposed with a second seal ring, and
the material circular plate holder is hermetically connected to the kit body to define a closed extraction and detection reaction space by the second seal ring.

5. The sample processing and detecting kit with the material transfer structure according to claim 1, wherein:
the sample processing plastic tube member is disposed with multiple reagent storage chambers and a reaction chamber, and
the reagent storage chambers and the reaction chamber define a closed area through the sealing film.

6. The sample processing and detecting kit with the material transfer structure according to claim 1, wherein:
the detection reaction plastic tube member comprises a plurality of detection reaction chambers, and
inner sides of the plurality of detection reaction chambers store detection reaction reagents or detection reaction lyophilized powders configured to complete a detection reaction of one or more items.

7. The sample processing and detecting kit with the material transfer structure according to claim 1, wherein:
the material transfer structure comprises a hollow rotatable shaft,
the pipette tip extends and overhangs from an end of the hollow rotatable shaft, and
the pipette tip comprises the hollow pointed structure for piercing the sealing film.

8. The sample processing and detecting kit with the material transfer structure according to claim 7, wherein:
the hollow rotatable shaft comprises a first through hole,
a first end of the first through hole is in communication with the pipette tip,
a second end of the first through hole penetrates the hollow rotatable shaft and abuts the silicone seal disposed in the rotatable connection structure, and
an anti-backflow mechanism is disposed on a joint of the hollow rotatable shaft and the pipette tip.

9. The sample processing and detecting kit with the material transfer structure according to claim 1, wherein the rotatable connection structure is engaged with an external device to enable the hollow rotor to rotate and to enable liquid to be sucked in and to be blown out.

10. A sample processing and detecting kit with a material transfer structure, comprising:
a kit body defined by an outer shell,
a material plate holder, and
the material transfer structure, wherein:
a top outer shell of the kit body comprises an opening-and-closing sample port,
a middle of the material plate holder is disposed with a circular hole,
a periphery of the circular hole is disposed with a waste liquid storage chamber, a first lock groove, a second lock groove, and a sealing hole,
a sample processing plastic tube member is hermetically disposed in the first lock groove,
a detection reaction plastic tube member is hermetically disposed in the second lock groove,
the material transfer structure comprises a hollow rotor disposed with a pipette tip, a rotatable connection structure, and a first seal ring,
an upper end portion of the hollow rotor is disposed with a hollow pointed structure for piercing a sealing film,
the material plate holder is disposed in the kit body,
the material plate holder is rotatably connected to the material transfer structure through the circular hole,
a rotatable connection structure surrounds a bottom end or an upper portion of the hollow rotor,
a silicone seal disposed below the bottom end of the hollow rotor is disposed in the rotatable connection structure,
the first seal ring surrounds an outer side of the hollow rotor,
the first seal ring is disposed in the circular hole,
an upper portion of the material transfer structure is hermetically disposed in the kit body through the first seal ring,
the rotatable connection structure drives the hollow rotor to rotate relative to the material plate holder, and
the hollow rotor is configured to move upward and downward relative to the material plate holder.

11. The sample processing and detecting kit with the material transfer structure according to claim 10, wherein:
the hollow rotor comprises a hollow rotatable shaft, and
the pipette tip extends and suspends from the upper portion of the hollow rotatable shaft.

12. The sample processing and detecting kit with the material transfer structure according to claim 11, wherein:
the hollow rotatable shaft is disposed with a first through hole,
first end of the first through hole is in communication with the pipette tip,
a second end of the first through hole penetrates the hollow rotatable shaft and abuts the silicone seal disposed in the rotatable connection structure, and
an inner side of the hollow rotatable shaft is disposed with a filter at a joint of the hollow rotatable shaft and the pipette tip.

13. The sample processing and detecting kit with the material transfer structure according to claim 10, wherein:
a reinforcing rib is disposed in the first lock groove, and
the sample processing plastic tube member is clamped in the first lock groove.

14. The sample processing and detecting kit with the material transfer structure according to claim 10, wherein:
a reinforcing rib is disposed in the second lock groove to define a plurality of lock groove units,
the detection reaction plastic tube member comprises a plurality of detection reaction chambers, and
each of the plurality of detection reaction chambers are clamped in a corresponding one of the plurality of lock groove units.

15. The sample processing and detecting kit with the material transfer structure according to claim 14, wherein the sealing hole is an individual tube clamped in one of the plurality of lock groove units or a sample processing tube of the sample processing plastic tube member.

16. The sample processing and detecting kit with a material transfer structure according to claim 10, wherein:
the rotatable connection structure is disposed on the upper end portion of the hollow rotor,
the pipette tip is disposed below the rotatable connection structure,
the kit body comprises an assembly hole, and
the upper end portion of the hollow rotor penetrates out of the assembly hole of the kit body to surround the rotatable connection structure.
